(19) United States
(12) Patent Application Publication
(10) Pub. No.: US 2026/0191700 A1
(43) Pub. Date: Jul. 9, 2026

(54) Absorbent Garment

(71) Applicant: Shenzhen Lute Jiacheng Supply Chain Management Co., Ltd., Shenzhen (CN)

(72) Inventors: Lan Li, Shenzhen (CN); Qian Feng, Shenzhen (CN); Ping Wang, Shenzhen (CN)

(21) Appl. No.: 19/555,928

(22) Filed: Mar. 4, 2026

(30) Foreign Application Priority Data
Dec. 2, 2025 (CN) 202522563620.2

(51) Int. Cl. A61F 13/49; A61F 13/494

(52) U.S. Cl. CPC A61F 13/49006; A61F 13/49406

(57) ABSTRACT

The present application discloses an absorbent garment comprising a panty body; a base layer joined to the panty body at least on both sides of the crotch region, wherein the base layer and the panty body enclose an accommodation space, the base layer is configured to face the wearer's crotch when worn, and the base layer comprises a first zone and a second zone, the first zone being located at a middle position of the crotch region, the second zone being located at side positions of the crotch region, and a liquid absorption rate per unit area of the first zone being greater than a liquid absorption rate per unit area of the second zone; and an absorption layer located within the accommodation space and joined to the base layer or the panty body.

### CROSS-REFERENCE TO RELATED APPLICATIONS

The present application claims priority to Chinese Patent Application No. 202522563620.2, filed on December 2, 2025, the entire contents of which are incorporated herein by reference.

### TECHNICAL FIELD

The present application relates to the technical field of maternal and baby products, particularly to a absorbent garment.

### BACKGROUND

In recent years, absorbent garments have gained increasing acceptance as a new type of product. Typically used by women during menstruation and also available for individuals with incontinence, these garments are designed to absorb bodily fluids and prevent leakage. Unlike disposable absorbent products such as sanitary pads or diapers, absorbent garments are reusable and washable clothing items.

An absorbent garment mainly consists of a panty body and an absorbent assembly. Side leakage of bodily fluids is one of the issues that need to be addressed in absorbent garments, as whether leakage occurs directly affects the wearer's experience.

As disclosed in U.S. Patent Application No. US 16/567,212, one existing approach involves bonding the absorbent assembly to the panty body via an elastic adhesive film. The elasticity at the sides causes the crotch region to tighten, thereby attempting to prevent side leakage and fully contain fluids within the absorbent assembly. However, this method suffers from poor breathability, and the garment tends to dry slowly after washing.

As disclosed in U.S. Patent Application No. US 17/475,690, another existing approach employs an elastic-bound stitching method. Here, the sides of the crotch region of the panty body wrap around the absorbent assembly to form elastic bindings, which are then stitched to the absorbent assembly. In this design, the stitching faces and is exposed to the wearer's crotch. Furthermore, due to the elasticity of the binding, the pressure generated presses the stitching tightly against the skin of the wearer's crotch, resulting in reduced wearing comfort.

In summary, traditional absorbent garments rely on physical tightening to prevent side leakage, for example, by enhancing elasticity at both sides of the crotch region in an attempt to create a seal. While this approach can, to some extent, block liquid that has already spread to the edges, it often comes at the cost of wearing comfort. Excessive tightness and constriction can cause continuous pressure and discomfort for the wearer, making it particularly unsuitable for extended wear during nighttime.

Therefore, there is a need to provide an absorbent garment to address the aforementioned technical issues.

### SUMMARY

In view of the shortcomings existing in the prior art, the present application provides an absorbent garment capable of resolving the discomfort issues caused by the side leakage prevention features of existing absorbent garments.

In order to solve the above technical problem, one embodiment of this application proposes an absorbent garment comprising: a panty body having an upper edge configured to surround a wearer's waist when worn, two leg openings, and a crotch region located between the two leg openings; a base layer joined to the panty body at least on both sides of the crotch region, wherein the base layer and the panty body enclose an accommodation space, the base layer is configured to face the wearer's crotch when worn, and the base layer comprises a first zone and a second zone, the first zone located at a middle portion of the crotch region and the second zone located at side portions of the crotch region, wherein a liquid absorption rate per unit area of the first zone is greater than a liquid absorption rate per unit area of the second zone; and an absorption layer disposed within the accommodation space and joined to the base layer or the panty body. In one embodiment, the base layer is joined to both sides of the crotch region of the panty body by stitches or by an adhesive bond.

In one embodiment, the first zone is configured to allow liquid to permeate therethrough to the absorption layer, and wherein the second zone is at least partially formed of a waterproof material configured to inhibit lateral diffusion of liquid and to block liquid from permeating to the absorption layer.

In one embodiment, the first zone comprises a unidirectional moisture-wicking fabric configured to allow liquid to permeate unidirectionally from the wearer's body toward the absorption layer, and wherein the second zone comprises a stack having a hydrophobic base layer and a waterproof adhesive layer, the hydrophobic base layer being disposed closer to the wearer's skin than the waterproof adhesive layer.

In one embodiment, the first zone is a first portion of the base layer, wherein the second zone is a second portion of the base layer, wherein the first portion has a third edge, wherein the second portion has a fourth edge adjacent to the third edge, and wherein the third edge and the fourth edge extend into the accommodation space and are joined together within the accommodation space.

In one embodiment, the fourth edge of the second portion is folded and extends into the accommodation space.

In one embodiment, the third edge and the fourth edge are joined by stitches or by an adhesive.

In one embodiment, further comprising a panty body waterproof layer disposed on at least a portion of a surface of the panty body that faces the accommodation space.

In one embodiment, a width of the absorption layer is less than a width of the crotch region, and wherein edges of the absorption layer are spaced inward from edges of the crotch region.

In one embodiment, the absorption layer is contained within a projected area of the first zone.

Another embodiment provided by the present application is: an absorbent garment comprising: a panty body having an upper edge configured to surround a wearer's waist when worn, two leg openings, and a crotch region located between the two leg openings; a base layer joined to the panty body at least at a portion of the crotch region, the base layer being configured to face the wearer's crotch when worn, and the base layer comprising a first zone and a second zone, the first zone located at a middle portion of the crotch region and the second zone located at side portions of the crotch region, wherein a liquid absorption rate per unit area of the first zone is greater than a liquid absorption rate per unit area of the second zone; and an absorption layer joined to the base layer or the panty body.

In one embodiment, the first zone is configured to allow liquid to permeate therethrough to the absorption layer, and wherein the second zone is at least partially formed of a waterproof material configured to inhibit lateral diffusion of liquid and to block liquid from permeating to the absorption layer.

In one embodiment, the first zone comprises a unidirectional moisture-wicking fabric configured to allow liquid to permeate unidirectionally from the wearer's body toward the absorption layer, and wherein the second zone comprises a stack having a hydrophobic base layer and a waterproof adhesive layer, the hydrophobic base layer being disposed closer to the wearer's skin than the waterproof adhesive layer.

In one embodiment, a width of the absorption layer is less than a width of the crotch region, and wherein edges of the absorption layer are spaced inward from edges of the crotch region.

In one embodiment, the absorption layer is contained within a projected area of the first zone.

Yet another embodiment provided by the present application is: an absorbent garment comprising: a panty body having an upper edge configured to surround a wearer's waist when worn, two leg openings, and a crotch region located between the two leg openings; a base layer joined to the panty body at least at a portion of the crotch region, the base layer being configured to face the wearer's crotch when worn, and the base layer comprising a first zone and a second zone, wherein a liquid absorption rate per unit area of the first zone is greater than a liquid absorption rate per unit area of the second zone, wherein the first zone occupies more than 90% of a total area of the base layer, and wherein the second zone occupies less than 10% of the total area of the base layer; and an absorption layer joined to the base layer or the panty body.

In one embodiment, the first zone is configured to allow liquid to permeate therethrough to the absorption layer, and wherein the second zone is at least partially formed of a waterproof material configured to inhibit lateral diffusion of liquid and to block liquid from permeating to the absorption layer.

In one embodiment, the first zone comprises a unidirectional moisture-wicking fabric configured to allow liquid to permeate unidirectionally from the wearer's body toward the absorption layer, and wherein the second zone comprises a stack having a hydrophobic base layer and a waterproof adhesive layer, the hydrophobic base layer being disposed closer to the wearer's skin than the waterproof adhesive layer.

In one embodiment, a width of the absorption layer is less than a width of the crotch region, and wherein edges of the absorption layer are spaced inward from edges of the crotch region.

In one embodiment, the absorption layer is contained within a projected area of the first zone.

The absorbent garment according to an embodiment of the present application comprises: a panty body having an upper edge configured to surround a wearer's waist when worn, two leg openings configured to allow the wearer's legs to pass through, and a crotch region located between the two leg openings; a base layer joined to the panty body at least on both sides of the crotch region, wherein the base layer and the panty body enclose an accommodation space, the base layer is configured to face the wearer's crotch when worn, and the base layer comprises a first zone and a second zone, the first zone being located at a middle position of the crotch region, the second zone being located at side positions of the crotch region, and a liquid absorption rate per unit area of the first zone being greater than a liquid absorption rate per unit area of the second zone; and an absorption layer located within the accommodation space and joined to the base layer or the panty body. In this manner, the present application departs from the passive leakage prevention approach of mere containment, adopting instead an active management strategy that combines guidance and interception. The centrally located first zone, with its high absorption rate, rapidly draws liquid away from the point of contact and directs it toward the underlying absorption layer, reducing the opportunity for liquid to remain on the skin surface or spread laterally. Simultaneously, the second zones on both sides act as low-absorption barriers. Their physical properties effectively prevent liquid from spreading laterally across their surfaces, confining the liquid within the central, high-efficiency absorption channel. The synergistic mechanism of these two aspects eliminates the possibility of liquid flowing toward the inner thighs and causing side leakage at the source of the liquid flow path. Consequently, intelligent and reliable side leakage prevention is achieved while ensuring wearing comfort.

### BRIEF DESCRIPTION OF THE DRAWINGS

To further clarify the technical solutions in the embodiments of this application or the prior art, a brief introduction to the drawings that need to be used in the description of the embodiments or prior art will be provided below. It is obvious that the drawings described below are merely some embodiments of this application, and for those skilled in the art, additional drawings may be obtained based on the structures shown in these drawings without requiring inventive effort.

FIG. 1 is a schematic front structural diagram of an absorbent garment according to an embodiment of the present application.

FIG. 2 is a schematic structural diagram of the inner side of the crotch region of the absorbent garment according to an embodiment of the present application.

FIG. 3 is a schematic cross-sectional structural diagram of the absorbent garment taken along the crotch region according to an embodiment of the present application.

FIG. 4 is a schematic diagram of a layered structure of the second zone of the base layer in the absorbent garment according to an embodiment of the present application.

The realization, functional characteristics, and advantages of the objectives of this application will be further explained in combination with the embodiments, with reference to the drawings.

### DETAILED DESCRIPTION OF THE EMBODIMENTS

In this application, the terms "provided," "disposed," and "connected" should be broadly understood. For example, they can refer to fixed connections, detachable connections, or integrated structures. They may indicate mechanical connections or electrical connections, direct connections, or indirect connections through an intermediary, or they may refer to internal communication between two devices, components, or parts. A person skilled in the art can understand the specific meaning of these terms in this application according to the context.

The terms "center," "longitudinal," "transverse," "length," "width," "thickness," "top," "bottom," "front," "back," "left," "right," "vertical," "horizontal," "inner," "outer," "radial," and "circumferential" indicate directional or positional relationships based on the orientation or positional relationships shown in the figures. These terms are used solely to simplify and clarify the description of the present application and should not be interpreted as limiting the invention to a specific orientation, construction, or operation.

Additionally, the terms "first" and "second" are used only for descriptive purposes and should not be understood as indicating relative importance or implying the number of features. Therefore, a "first" or "second" feature may explicitly or implicitly include one or more of that feature. In the description of this application, "multiple" means at least two, such as two or three, unless otherwise explicitly specified.

Moreover, the above terms, in addition to indicating directional or positional relationships, may also have other meanings. For example, the term "top" may also refer to attachment or connection relationships. A person skilled in the art can understand these terms according to the specific context.

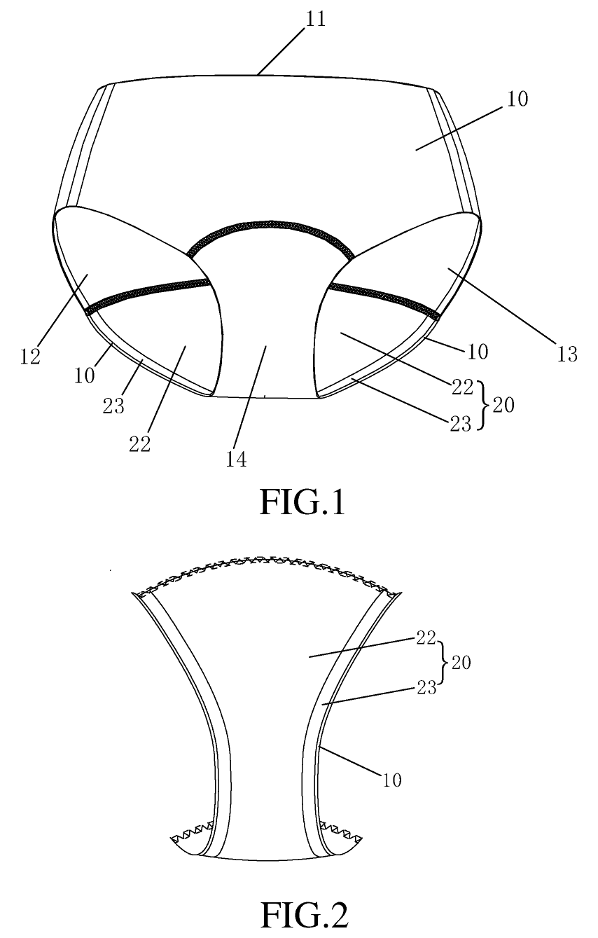

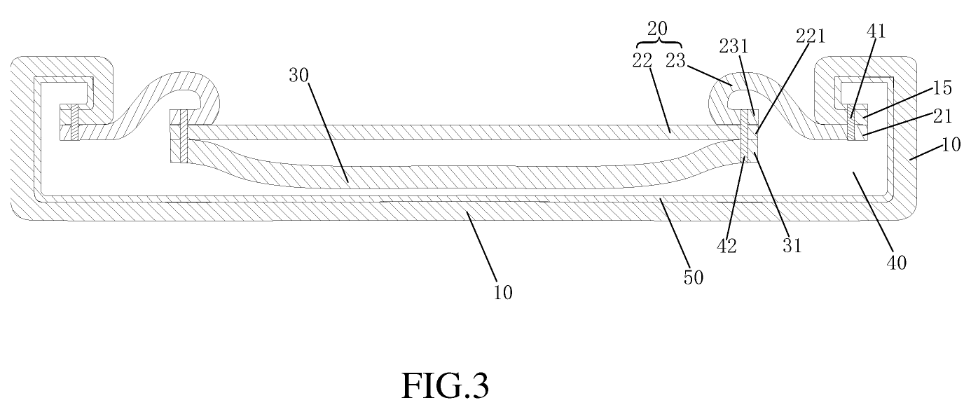

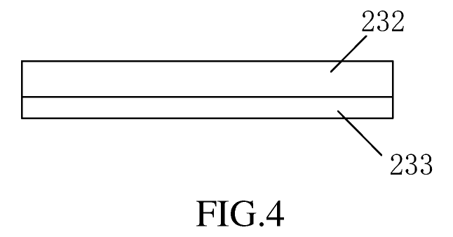

Please refer to FIGS. 1-4. FIG. 1 is a schematic front structural diagram of an absorbent garment according to an embodiment of the present application. FIG. 2 is a schematic structural diagram of the inner side of a crotch region of the absorbent garment according to an embodiment of the present application. FIG. 3 is a schematic cross-sectional structural diagram of the absorbent garment taken along the crotch region according to an embodiment of the present application. FIG. 4 is a schematic diagram of a layered structure of a second zone of a base layer in the absorbent garment according to an embodiment of the present application.

As shown in FIGS. 1, 2, and 3, in an embodiment of the present application, the absorbent garment may comprise a panty body 10, a base layer 20, and an absorption layer 30.

As shown in FIG. 1, the panty body 10 has an upper edge 11 configured to surround a wearer's waist when worn, two leg openings 12, 13 configured to allow the wearer's legs to pass through, and a crotch region 14 located between the two leg openings 12, 13.

As shown in FIG. 1, the panty body 10 employs a breathable, moisture-wicking functional fabric at least in the crotch region 14, such as Modal, Lycra, or cotton material with a honeycomb weave, to further enhance overall comfort.

As shown in FIGS. 1, 2, and 3, the base layer 20 is joined to the panty body 10 at least on both sides of the crotch region 14, and the base layer 20 and the panty body 10 together enclose an accommodation space 40. The base layer 20 is configured to face the wearer's crotch when worn. In one embodiment, the base layer 20 is joined to the panty body 10 at least on the left and right sides of the crotch region 14. The "both sides of the crotch region 14" refer to the left and right sides respectively adjacent to the left and right leg openings 12, 13.

As shown in FIG. 3, specifically, in one embodiment, the panty body 10 has a first edge 15 on one side of the crotch region 14, and the base layer 20 has a corresponding second edge 21. The first edge 15 and the second edge 21 extend into the accommodation space 40 and are stitched together within the accommodation space 40 by a seam 41. The "side edge of the crotch region 14" refers to one of the left and right sides. In this embodiment, both sides of the crotch region 14 are joined using the aforementioned identical stitching method. It should be understood that, in some embodiments, only one side may utilize the aforementioned stitching method, while the other side may employ a different stitching method or an adhesive bonding method, which is not limited in the embodiments of the present application. In other embodiments, the manner in which the base layer 20 is joined to the panty body 10 at least on both sides of the crotch region 14 may be other stitching methods or adhesive bonding using glue, which is not limited in the embodiments of the present application.

As shown in FIG. 3, the absorption layer 30 is located within the accommodation space 40 and is joined to the base layer 20. The absorption layer 30 may be formed by compositing one or more materials selected from terry cloth, dust-free paper, fluff pulp, superabsorbent polymer (SAP), and hydrophilic non-woven fabric. In some embodiments, the absorption layer 30 may also be joined to the panty body 10, which is not limited in the embodiments of the present application.

As shown in FIG. 3, in one embodiment, the first edge 15 of the panty body 10 is folded and then extends into the accommodation space 40. This ensures that the outer surface at the joint of the panty body is smooth, preventing discomfort in the crotch region. As shown in FIG. 3, in one embodiment, the seam 41 is concealed within the accommodation space 40. By stitching the first edge 15 of the panty body 10 and the second edge 21 of the base layer 20 together within the accommodation space 40, the seam 41 is fully contained and concealed within the accommodation space 40. This internally enclosed stitching structure offers multiple benefits: Firstly, it ensures complete isolation between the stitched area and the wearer's skin, fundamentally eliminating potential friction from the seam and enhancing skin-contact comfort. Secondly, compared to large-area adhesive bonding, the seam itself does not form an airtight barrier, preserving breathability in this area. This allows for faster drying after washing and improves the feeling of dryness during wear. Finally, the mechanical strength and wash durability of the stitched connection are significantly superior to adhesive bonding, effectively overcoming the issue of adhesive failure.

As shown in FIGS. 1, 2, and 3, in one embodiment, the base layer 20 comprises a first zone 22 and a second zone 23. The first zone 22 is located at a middle position of the crotch region 14, and the second zone 23 is located at side positions of the crotch region 14. A liquid absorption rate per unit area of the first zone 22 is greater than a liquid absorption rate per unit area of the second zone 23. In this manner, the present application departs from the passive leakage prevention approach of mere containment, adopting instead an active management strategy that combines guidance and interception. The centrally located first zone 22, with its high absorption rate, rapidly draws liquid away from the point of contact and directs it toward the underlying absorption layer, reducing the opportunity for liquid to remain on the skin surface or spread laterally. Simultaneously, the second zones 23 on both sides act as low-absorption barriers. Their physical properties effectively prevent liquid from spreading laterally across their surfaces, confining the liquid within the central, high-efficiency absorption channel. The synergistic mechanism of these two aspects eliminates the possibility of liquid flowing toward the inner thighs and causing side leakage at the source of the liquid flow path, thereby achieving intelligent and reliable side leakage prevention. Through the above configuration, due to the liquid wicking effect, liquid is concentrated and absorbed by the first zone 22 at the middle position of the crotch region 14 and directed to the absorption layer 30, thereby reducing the risk of side leakage. In one embodiment, a coverage area of the first zone 22 accounts for more than 90% of an entire coverage area of the base layer 20, and a coverage area of the second zone 23 accounts for less than 10% of the entire coverage area of the base layer 20. The number of second zones 23 may be two, and the two second zones 23 may be respectively located on the left and right sides of the first zone 22.

As shown in FIGS. 1, 2, and 3, in one embodiment, the first zone 22 is configured to allow liquid to permeate to the absorption layer 30, and the second zone 23 is configured to restrict liquid permeation and/or lateral diffusion.

Specifically, in one embodiment, the first zone 22 is configured to allow liquid to permeate therethrough and flow toward the absorption layer 30. The second zone 23 is at least partially made of a waterproof material and is configured to inhibit lateral diffusion of liquid and to block liquid from permeating to the absorption layer 30. The base layer 20 employs a zoned design, the core of which lies in directional liquid diversion and three-dimensional leakage prevention. The centrally located first zone 22 serves as a diversion zone. It comprises a unidirectional moisture-wicking fabric 221, which can quickly absorb liquids such as menstrual fluid from the skin surface and direct it to the underlying absorption layer 30. Meanwhile, leveraging its unidirectional moisture-wicking property, it effectively reduces liquid re-wetting, maintaining relative dryness on the skin-contact surface.

As shown in FIGS. 1, 2, and 3, in one embodiment, the first zone 22 is a unidirectional moisture-wicking fabric 22 configured to allow liquid to permeate unidirectionally from the wearer's body to the absorption layer 30. In a specific example, the unidirectional moisture-wicking fabric 22 is a lightweight knitted fabric with a weight ranging between 95 g/m² and 110 g/m². Preferably, the unidirectional moisture-wicking fabric 22 has a rib jacquard structure formed from hydrophobic polymer yarns (e.g., polyester), which generates a capillary effect through its specific knit construction to achieve the unidirectional moisture-wicking function. It is understood that the unidirectional moisture-wicking fabric 22 is not limited to the aforementioned specific composition. It may be any functional fabric capable of achieving unidirectional liquid transport via capillary action, differential wettability, or a combination thereof. For example, the unidirectional moisture-wicking fabric 22 may be a double-layer composite structure, with its skin-contact layer comprising hydrophobic fibers such as fine-denier polyester or polypropylene, and its outer layer comprising hydrophilic fibers such as cotton or rayon. Alternatively, the unidirectional moisture-wicking fabric 22 may also be obtained by applying differential hydrophilic/hydrophobic finishing treatments to the inner and outer surfaces of a single-layer fabric.

As shown in FIG. 4, the second zone 23 comprises a stack having a hydrophobic base layer 232 and a waterproof adhesive layer 233, the hydrophobic base layer 232 being disposed closer to the wearer's skin than the waterproof adhesive layer233. For example, the second zone 23 comprises a hydrophobic base layer 232 and a waterproof adhesive layer 233 arranged in a laminate, wherein the hydrophobic base layer 232 is disposed closer to the wearer's skin than the waterproof adhesive layer 233. The second zones 23 located on both sides constitute side-leakage prevention zones. Their laminated structure (the hydrophobic base layer 232 and the waterproof adhesive layer 233) works in concert: the hydrophobic base layer 232 near the skin effectively prevents liquid from spreading onto its surface, forcing liquid to concentrate towards the central diversion zone; meanwhile, the waterproof adhesive layer 233 acts as a secondary barrier, preventing any potentially permeating liquid from further wetting the panty body 10. This mechanism of zoned, cooperative operation significantly enhances the reliability of side-leakage prevention while ensuring efficient absorption. The hydrophobic base layer 232 is a non-woven fabric or textile possessing hydrophobic properties. The waterproof adhesive layer 233 is a continuous waterproof barrier layer, its material selected from thermoplastic polyurethane (TPU) film, polyethylene (PE) film, or waterproof polyurethane (PU) coating. This layer is bonded to the hydrophobic base layer 232 via a thermal lamination process, forming a barrier that, due to its dense, non-porous microstructure, completely blocks liquid permeation to the panty body 10.

As shown in FIGS. 1-3, in one embodiment, the first zone 22 is a first portion 22 of the base layer 20, and the second zone 23 is a second portion 23 of the base layer 20. The first portion 22 has a third edge 221, and the second portion 23 has a fourth edge 231 adjacent to the third edge 221. The third edge 221 and the fourth edge 231 extend into the accommodation space 40 and are joined together within the accommodation space 40. In other embodiments, the base layer 20 may be an integrated whole.

As shown in FIG. 3, in one embodiment, the fourth edge 231 of the second portion 23 is folded and then extends into the accommodation space 40. This ensures a smooth outer surface at the joint of the panty body, preventing discomfort in the crotch region. As shown in FIG. 3, in one embodiment, the third edge 221 and the fourth edge 231 are joined by a seam 42. In other embodiments, the third edge 221 and the fourth edge 231 may also be joined by an adhesive.

As shown in FIG. 3, in one embodiment, the absorbent garment further comprises a panty body waterproof layer 50. The panty body waterproof layer 50 is disposed on at least a portion of the surface of the panty body 10 facing the accommodation space 40. Optionally, the panty body waterproof layer 50 covers the entire inner surface of the crotch region. As shown in FIG. 3, in one embodiment, a width of the absorption layer 30 is less than a width of the crotch region 14, and edges of the absorption layer 30 do not extend to edges of the crotch region 14. Through this configuration, by not extending the absorption layer 30 to the edges of the crotch region 14, the risk of side leakage is reduced.

As shown in FIG. 3, in one embodiment, the absorption layer 30 and the first portion 22 are jointly joined to the second portion 23. The absorption layer 30 has a fifth edge 31. The third edge 221, the fourth edge 231, and the fifth edge 31 extend into the accommodation space 40 and are joined together within the accommodation space 40.

As shown in FIG. 3, in one embodiment, the third edge 221, the fourth edge 231, and the fifth edge 31 are joined by a seam 42 within the accommodation space 40.

As shown in FIG. 3, in one embodiment, an extent of the absorption layer 30 does not exceed a coverage area of the first zone 22.

The absorbent garment according to an embodiment of the present application comprises: a panty body having an upper edge configured to surround a wearer's waist when worn, two leg openings configured to allow the wearer's legs to pass through, and a crotch region located between the two leg openings; a base layer joined to the panty body at least on both sides of the crotch region, wherein the base layer and the panty body enclose an accommodation space, the base layer is configured to face the wearer's crotch when worn, and the base layer comprises a first zone and a second zone, the first zone being located at a middle position of the crotch region, the second zone being located at side positions of the crotch region, and a liquid absorption rate per unit area of the first zone being greater than a liquid absorption rate per unit area of the second zone; and an absorption layer located within the accommodation space and joined to the base layer or the panty body. In this manner, the present application departs from the passive leakage prevention approach of mere containment, adopting instead an active management strategy that combines guidance and interception. The centrally located first zone, with its high absorption rate, rapidly draws liquid away from the point of contact and directs it toward the underlying absorption layer, reducing the opportunity for liquid to remain on the skin surface or spread laterally. Simultaneously, the second zones on both sides act as low-absorption barriers. Their physical properties effectively prevent liquid from spreading laterally across their surfaces, confining the liquid within the central, high-efficiency absorption channel. The synergistic mechanism of these two aspects eliminates the possibility of liquid flowing toward the inner thighs and causing side leakage at the source of the liquid flow path, thereby achieving intelligent and reliable side leakage prevention.

While certain features of the embodiments of the subject matter claimed have been illustrated and described herein, many modifications, substitutions, changes, and equivalents will now occur to those skilled in the art. Moreover, although several functional modules and their relationships have been described in detail, it is believed by those skilled in the art that some operations can be performed without using other functional modules, or other functions or relationships between functions can be established while still conforming to the subject matter claimed. Therefore, it is intended that the appended claims encompass all such modifications and variations that fall within the true spirit and scope of the embodiments of the claimed subject matter.

The above description only provides specific embodiments of the present application. It should be noted that for ordinary technicians in the technical field, without departing from the principles of the present application, several improvements and embellishments can also be made, which should also be regarded as falling within the protection scope of the present application.

What is claimed is:

1. An absorbent garment comprising:
a panty body having an upper edge configured to surround a wearer's waist when worn, two leg openings, and a crotch region located between the two leg openings;
a base layer joined to the panty body at least on both sides of the crotch region, wherein the base layer and the panty body enclose an accommodation space, the base layer is configured to face the wearer's crotch when worn, and the base layer comprises a first zone and a second zone, the first zone located at a middle portion of the crotch region and the second zone located at side portions of the crotch region, wherein a liquid absorption rate per unit area of the first zone is greater than a liquid absorption rate per unit area of the second zone; and
an absorption layer disposed within the accommodation space and joined to the base layer or the panty body.

2. The absorbent garment of claim 1, wherein the base layer is joined to both sides of the crotch region of the panty body by stitches or by an adhesive bond.

3. The absorbent garment of claim 1, wherein the first zone is configured to allow liquid to permeate therethrough to the absorption layer, and wherein the second zone is at least partially formed of a waterproof material configured to inhibit lateral diffusion of liquid and to block liquid from permeating to the absorption layer.

4. The absorbent garment of claim 1, wherein the first zone comprises a unidirectional moisture-wicking fabric configured to allow liquid to permeate unidirectionally from the wearer's body toward the absorption layer, and wherein the second zone comprises a stack having a hydrophobic base layer and a waterproof adhesive layer, the hydrophobic base layer being disposed closer to the wearer's skin than the waterproof adhesive layer.

5. The absorbent garment of claim 1, wherein the first zone is a first portion of the base layer, wherein the second zone is a second portion of the base layer, wherein the first portion has a third edge, wherein the second portion has a fourth edge adjacent to the third edge, and wherein the third edge and the fourth edge extend into the accommodation space and are joined together within the accommodation space.

6. The absorbent garment of claim 5, wherein the fourth edge of the second portion is folded and extends into the accommodation space.

7. The absorbent garment of claim 5, wherein the third edge and the fourth edge are joined by stitches or by an adhesive.

8. The absorbent garment of claim 1, further comprising a panty body waterproof layer disposed on at least a portion of a surface of the panty body that faces the accommodation space.

9. The absorbent garment of claim 1, wherein a width of the absorption layer is less than a width of the crotch region, and wherein edges of the absorption layer are spaced inward from edges of the crotch region.

10. The absorbent garment of claim 1, wherein the absorption layer is contained within a projected area of the first zone.

11. An absorbent garment comprising:
a panty body having an upper edge configured to surround a wearer's waist when worn, two leg openings, and a crotch region located between the two leg openings;
a base layer joined to the panty body at least at a portion of the crotch region, the base layer being configured to face the wearer's crotch when worn, and the base layer comprising a first zone and a second zone, the first zone located at a middle portion of the crotch region and the second zone located at side portions of the crotch region, wherein a liquid absorption rate per unit area of the first zone is greater than a liquid absorption rate per unit area of the second zone; and
an absorption layer joined to the base layer or the panty body.

12. The absorbent garment of claim 11, wherein the first zone is configured to allow liquid to permeate therethrough to the absorption layer, and wherein the second zone is at least partially formed of a waterproof material configured to inhibit lateral diffusion of liquid and to block liquid from permeating to the absorption layer.

13. The absorbent garment of claim 11, wherein the first zone comprises a unidirectional moisture-wicking fabric configured to allow liquid to permeate unidirectionally from the wearer's body toward the absorption layer, and wherein the second zone comprises a stack having a hydrophobic base layer and a waterproof adhesive layer, the hydrophobic base layer being disposed closer to the wearer's skin than the waterproof adhesive layer.

14. The absorbent garment of claim 11, wherein a width of the absorption layer is less than a width of the crotch region, and wherein edges of the absorption layer are spaced inward from edges of the crotch region.

15. The absorbent garment of claim 11, wherein the absorption layer is contained within a projected area of the first zone.

16. An absorbent garment comprising:
a panty body having an upper edge configured to surround a wearer's waist when worn, two leg openings, and a crotch region located between the two leg openings;
a base layer joined to the panty body at least at a portion of the crotch region, the base layer being configured to face the wearer's crotch when worn, and the base layer comprising a first zone and a second zone, wherein a liquid absorption rate per unit area of the first zone is greater than a liquid absorption rate per unit area of the second zone, wherein the first zone occupies more than 90% of a total area of the base layer, and wherein the second zone occupies less than 10% of the total area of the base layer; and
an absorption layer joined to the base layer or the panty body.

17. The absorbent garment of claim 16, wherein the first zone is configured to allow liquid to permeate therethrough to the absorption layer, and wherein the second zone is at least partially formed of a waterproof material configured to inhibit lateral diffusion of liquid and to block liquid from permeating to the absorption layer.

18. The absorbent garment of claim 16, wherein the first zone comprises a unidirectional moisture-wicking fabric configured to allow liquid to permeate unidirectionally from the wearer's body toward the absorption layer, and wherein the second zone comprises a stack having a hydrophobic base layer and a waterproof adhesive layer, the hydrophobic base layer being disposed closer to the wearer's skin than the waterproof adhesive layer.

19. The absorbent garment of claim 16, wherein a width of the absorption layer is less than a width of the crotch region, and wherein edges of the absorption layer are spaced inward from edges of the crotch region.

20. The absorbent garment of claim 16, wherein the absorption layer is contained within a projected area of the first zone.
